(19) United States
(12) Patent Application Publication
(10) Pub. No.: US 2002/0082230 A1
(43) Pub. Date: Jun. 27, 2002

(54) Antisense oligomer

(76) Inventors: Amos Douvdevani, Beer Sheva (IL); Cidio Chaimovitz, Omer (IL)

Correspondence Address: BAKER & BOTTS, 30 ROCKEFELLER PLAZA, NEW YORK, NY, 10112

(21) Appl. No.: 09/849,014

(22) Filed: May 4, 2001

Related U.S. Application Data
(63) Continuation of application No. PCT/IL99/00589, filed on Nov. 4, 1999.

(30) Foreign Application Priority Data
Nov. 5, 1998 (IL) 126969
May 11, 1998 (IL) 126919

(51) Int. Cl. A61K 48/00; C07H 21/04; A61K 31/675; C07D 413/14

(52) U.S. Cl. 514/44; 536/23.5; 514/81; 544/81

(57) ABSTRACT

An antisense oligomer capable of inhibiting production of interleukin-15 (IL-15) by hybridizing to the mRNA of IL-15. Also disclosed is a pharmaceutical composition for treating diseases associated with the production of IL-15 comprising said antisense oligomer and a pharmaceutically acceptable carrier.

This application is a continuation of PCT/IL99/0589 filed Nov. 4, 1999 and published in English on May 18, 2000 as WO 00/28019, which claims priority to Israeli Patent Application No. 126,969, filed Nov. 5, 1998.

### FIELD OF THE INVENTION

This invention relates to antisense oligomers which interfere with the production of interleukin-15, and their use in the treatment of various diseases.

### BACKGROUND OF THE INVENTION

Cytokines are proteins involved in the mediation of regulatory and effector activities related to the immune response and cell proliferation. Numerous types of cytokines have been discovered during the past 20 years, including various interleukins (IL) such as IL-1, IL-2 and IL-6. Recently, a novel cytokine, IL-15, was defined. IL-15 is a potent T-cell growth factor and activator which stimulates the proliferation of the cytokine-dependent murine T cell line CTLL-2 (Tagaya, Y., Bamford, R. N., DeFiilippis, A. P., Waldmannn, T. A. Immunity (1996) 4:329-336). Although IL-15 shares many features with IL-2, these interleukins also differ in a number of aspects.

The cDNA of IL-15 encodes a 162 amino acid protein, of which 48 amino acids comprise a leader sequence. IL-15 is a member of the short chain, four α helix bundle cytokine family. Human IL-15 cDNA contains a 3 16-nucleotide 5′ untranslated region (UTR), a coding sequence of 486 nucleotides and a 400 nucleotide 3′ UTR (Grabstein, K. H., et al. Science (1 994) 264:965-968) (SEQ. ID. NO. 1).

A number of disease states and pathological conditions are mediated by T-cells. These include autoimmune disease, rheumatoid arthritis, graft versus host disease and organ transplant rejection. IL-15 was found to act as a stimulator of various pathological immune responses. For example, IL-15 was found to be actively transcribed in human renal allograft rejection (Pavlakis, M. et al. Transplantation (1996) 62:543-545). IL-15 was found to have a proinflammatory role in rheumatoid arthritis synovitis (McInnes, I. B. and Liew, F. Y. Immunology Today (1998) 19:75-79).

Antisense oligonucleotides are short, single-stranded nucleic acids which bind to a corresponding target RNA as a result of their complementary sequence. They have been suggested for use as a therapeutic agent which acts by binding and blocking the translation of the mRNA of pathological proteins. For example, the leukocyte adhesion molecule ICAM-1 is implicated in ischemic renal reperfusion injury, and antisense oligonucleotides for ICAM-1 were found to attenuate reperfusion injury and renal failure in the rat (Haller, H. et al Kidney International (1996) 50:473-480). The kidney was also found in other studies to be an excellent target for site-directed antisense therapy. In another example, rheumatoid synovial fibroblast proliferation induced by IL-1β was inhibited by IL-1β antisense (Morita, Y. et al. Arthritis & Rheumatism (1997) 40:1292-1297).

Attempts have been made to treat IL-15-associated diseases by interfering with the production of IL-15. WO 98/18812 discloses single-stranded oligonucleotides which, as the authors claim, are capable of forming a triplex DNA within the transcribed region of the IL-15 gene. The authors hypothesize that the oligonucleotides bind in an antiparallel orientation to the polypurine strand of the promoter region of the IL-IS gene. No experimental data is presented to support their claim that the oligonucleotides inhibit IL-15 gene expression. Furthermore, a screening of DNA databases carried out by the inventors has revealed that the disclosed oligonucleotides are completely non-specific and bind over 500 DNA sequences.

WO 96/26274 discloses antagonists of IL-15 in the form of IL-15 muteins. The muteins are created by additions, deletions or substitutions at key positions in the amino acid sequence of IL-15. These muteins are believed to compete with the binding of IL-15 to its receptor(s). Also disclosed are monoclonal antibodies to IL-15.

### SUMMARY OF THE INVENTION

The present invention provides novel therapies and compounds for treating inflammatory polyarthropathies like rheumatoid arthritis, organ and cell transplant rejections, inflammatory bowel disease and other immune mediated pathologies in which IL-15 plays a role. In these therapies, expression of IL-15 genes is inhibited, resulting in apoptosis of self-reacting T-cells and suppression of T-cell and other immune cell recruitment and activation. By this selective immune suppression, autoimmune-mediated pathological damage is prevented.

Thus, in a first aspect of the invention, there is provided an antisense oligomer capable of inhibiting production of interleukin-15 (IL-15) by hybridizing to the mRNA of IL-15. The oligomer of the invention is superior to previously disclosed antisense oligomers which bind DNA both in specificity and in activity.

In a second aspect of the invention, there is provided a method of inhibiting production of interleukin-15 (IL-15) by a cell comprising the following steps: (a) introducing an antisense oligomer as defined above into said cell; and (b) allowing said oligomer to hybridize to the mRNA of IL-15, thereby inhibiting the production of IL-15.

In a third aspect of the invention, there is provided a pharmaceutical composition for treating inflammatory polyarthropathies comprising an antisense oligomer capable of inhibiting production of interleukin-15 (IL-15) by hybridizing to the mRNA of IL-15, and a pharmaceutically acceptable carrier.

The oligomer of the invention may be an oligonucleotide or an oligonucleotide analog. In a preferred embodiment, the oligonucleotide is DNA. The oligonucleotide may be modified in the form of a phosphodiester, phosphorothioate, ethylphosphonate, methylphosphonate, or methylphosphonothioate oligonucleotide. In a preferred embodiment, the oligonucleotide is a phosphorothioate. The oligonucleotide should be at least 5 nucleotides in length in order to bind stabily to the mRNA. Preferably, the length of the oligonucleotide will be 5-50 nucleotides. Most preferably, the oligonucleotide will be approximately 20 nucleotides in length, in order to avoid undesired hybridizations. Longer lengths may permit partial, though active, interactions.

Oligonucleotide analogs which may be used in the invention include, but are not limited to, protein nucleic acid, morpholino, methylene linkage, boronated, and pteridine oligonucleotide analogs. The oligonucleotide analog may be linked at its 5′ end or at its 3′ end to an intercalator such as psoralen or acridine derivatives.

Also included in the invention are oligomers comprising the antisense oligomers of the invention, as well as fragments of the oligomers of the invention which retain the capability of inhibiting production of interleukin-15 (IL-15) by hybridizing to the mRNA of IL-15.

The preparation of an effective antisense oligomer entails using a nucleotide sequence which, on hybridizing to the mRNA of IL-15, will inhibit translation of the mRNA. Various considerations may be taken into account in the rational design of such an antisense oligomer. Rationales which may be used include the following:

1. Targeting the oligomer to a specific functional region of the mRNA, such as the ORF, the 5′-UTR, the 3′-UTR and the AU-rich region (all defined below in the Detailed Description section);

2. The two- and three-dimensional structures of the mRNA in the target region, and of the antisense oligomer;

3. The size of the target region and of the antisense oligomer;

4. The presence of specific nucleotide sequences in the oligomer which may cause undesirable side effects;

5. Homology of the target region with other RNA sequences;

6. The stability of the antisense molecule; and

7. The stability of the mRNA-antisense hybrid.

In addition, two or more different oligomers may be used in combination to obtain a synergistic effect. Rationales for choosing an effective combination may include the following:

1. That the two antisense molecules have differing mechanisms of IL-15 production inhibition;

2. That the distance between the target regions is significant;

3. That the mRNA-antisense complex is stabile; and

4. That the antisense oligomers do not hybridize with each other.

Examples of the above rationales will be given in the Detailed Description section below. The average skilled man of the art will be able to design antisense oligomer and oligomer combinations useful in the invention on the basis of the rationales listed above as well as on the basis of the examples described below.

The oligomers of the invention may be used for treating diseases associated with the production of IL-15 such as inflammatory polyarthropathies, e.g. as are found associated with rheumatoid arthritis, organ and cell transplant rejections, asthma, inflammatory bowel disease, lupus erythematosus and other immune mediated pathologies in which IL-15 plays a role. An example of an organ transplant whose prognosis may be improved by the present invention is kidney transplant.

Extensive literature exists with respect to the application of antisense oligomers to human therapeutics (e.g. Gewirtz, A. M. Current Opinion in Hematology (1998) 5:59-71). A number of techniques have been suggested for delivering the oligomers into their target cells and allowing them to reach their target in a bioavailable form. For example, cationic lipid formulations, liposomes, viral vectors and electroporation have been suggested for use in inserting the antisense into the cells. Results of clinical studies using antisense oligonucleotides have also been reported (e.g. Glover, J. M. et al. J. Pharmacol. Exp. Ther. (1997) 282:1173-80).

The pharmaceutical composition of the invention may be prepared comprising an oligomer according to the invention dispersed in a pharmaceutically acceptable carrier. Such carriers may include physiological saline and sterile water. The composition may be in the form of e.g. an injectable preparation, a spray, an ointment, cream, gel, tablets, or perfusion.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the complete sequence of the mRNA of human IL-15; and

FIG. 2 is a graphic illustration of the effect of oligomers of the invention on IL-15 production.

### DETAILED DESCRIPTION OF PREFERRED EMBODIMENTS

The following abbreviations will be used at times in the description:

ODN: Oligodeoxynucleotide

ORF: Open reading frame, the mRNA region that codes for the protein product. The region begins with an AUG start codon.

′5-UTR: ′5-untranslated region, the mRNA region in which the action of the ribosome is initiated.

′3-UTR: ′3-untranslated region, the mRNA region that is believed to regulate the stability of the mRNA molecule.

AU-rich region: A region in the ′3-UTR that confers mRNA instability.

′5-exonucleases: The family of nucleases that brings about the major non-specific degradation of ODN molecules.

CG: ODN molecules that contain one or more CG sequences and cause an increase in TNFα levels in mouse blood circulation.

GGGG: ODN molecules that contain this sequence bind the protein PLA₂.

Materials and Methods

Sequences

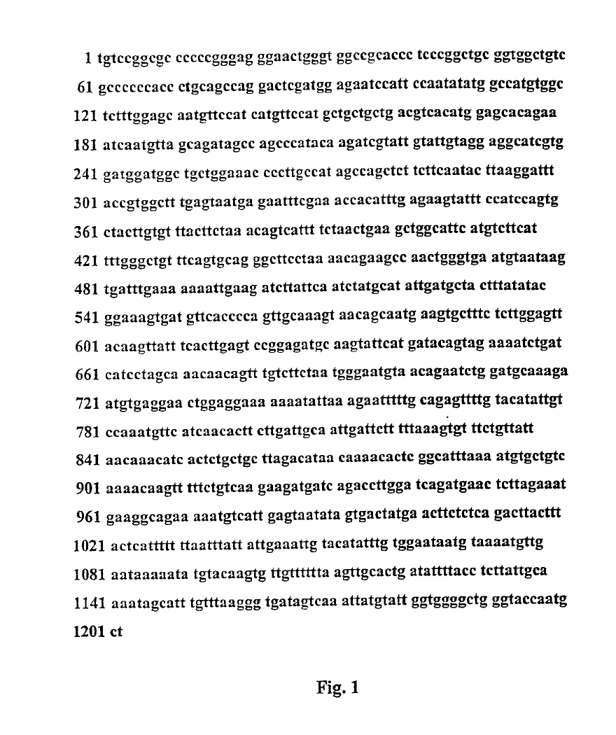

hIL-15-mRNA: The complete sequence (see FIG. 1) (SEQ. ID. NO. 1) was obtained from Genebank, accession code U14407.

hIL-15-mRNA configurations: The twelve most stable hIL-15-mRNA computed structures were predicted by the RNA folding software accessible at http ://www.ibc.wustl.edu/˜zuker/rna.

Statistics:

Data is presented as mean ± standard error of mean.

Cell Preparation

ODN inhibitory activity was determined using a primary culture of human renal proximal tubular cells obtained from healthy sections of kidneys that were removed from patients undergoing elective nephrectomy. Cells were grown to confluence in 24-well plates.

ODN Preparation

Unmodified ODN molecules were synthesized in an automatic DNA-synthesizer, solublized in water and stored at -20° C. ODN concentration was determined by a DNA-quantification kit (DNA Quik Srip, Kodak, New Haven USA).

Evaluation of IL-15 Production Inhibition

Cells were administered vehicle-free ODN via culture medium at 0 h, following medium replacement, and at 24 h, following washing of cells.

At 24 h 500 U/ml of IFNγ were added to cells for induction of IL-15 production. Cells were lysed at 48 h by NP-40. Protein levels were determined by specific ELISA. Cycloheximide, administered on an identical time course as ODN, was used as a reference for total non-specific inhibition of protein production.

### EXAMPLES

Individual ODN Preparations

ODN 1.

Sequence of ODN 1: ′5-ATA TGT ACA ATT TCA ATA AT-′3 (SEQ. ID. NO. 2)

Length of ODN: 20 nucleotides.

Target location of ODN on IL-15-mRNA (base numbering by Genebank): 1038-1057

Rational Behind ODN Selection:

1. The ODN is targeted to an AU-rich region in the IL-15-mRNA.

2. The computed structure of the IL-15-mRNA in this region is a single-strand loop positioned on a double-strand stem, separated from neighboring mRNA structures. This structure is identical in all IL-15-mRNA configurations.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in four separate experiments. In each experiment, human renal proximal tubular cells were incubated for 48 hours with 2 M ODN (double administration, 24 hours apart). The ODN inhibited inducible IL-15 production by 38.3%±10.2, and basal IL-15 production by 29.9%±8.4.

The specific activity of the ODN was demonstrated by repeating the above experiment with a mutated ODN sequence, in which four central nucleotides were replaced with nucleotides that are non-complementary to the IL-15-mRNA in this specific target site (′5-ATA TGT Agg caT TCA ATA AT-′3) (SEQ. ID. NO 3). The mutated-ODN inhibited inducible IL-15 production by 17.0%±3.0, and basal IL-15 production by 7.5%±0.5.

ODN 2.

Sequence of ODN: ′5-AAG AGC CAC ATG GCC ATA TA-′3 (SEQ. ID. NO. 4) p0 Length of ODN: 20 nucleotides.

Target location of ODN on IL-15-mRNA (base numbering by Genebank): 105-124

Rational Behind ODN Selection:

1. The ODN is targeted to the 5′-UTR of the IL-15-mRNA.

2. The computed structure of the IL-15-mRNA in this region is comprised of two single-strand loops in tandem, positioned on a double-strand stem and separated from neighboring mRNA structures. This structure is identical in all IL-15-mRNA configurations.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in a single experiment, in which human renal proximal tubular cells were incubated for 48 hours with 2 M ODN (double administration, 24 hours apart). The ODN inhibited inducible IL-15 production by 26.26%, and basal IL-15 production by 13.58%.

ODN 3.

Sequence of ODN: ′5-CAT TGA TTT CTG TGC TCC AT-′3 (SEQ. ID. NO. 5)

Length of ODN: 20 nucleotides.

Target location of ODN on IL-15-mRNA (base numbering by Genebank): 168-187

Rational Behind ODN Selection:

7. The ODN is targeted to the 5′-UTR of the IL-15-mRNA.

8. The computed structure of the IL-15-mRNA in this region is comprised of two single-strand loops in tandem, positioned on a double-strand stem and separated from neighboring mRNA structures. This structure is identical in all IL-15-mRNA configurations but two, of which one is comprised of one long single-strand loop.

9. The part of ODN that is aimed for IL-15-mRNA single-strand binding is longer than four nucleotides, and is positioned away from the 5′ end of the ODN, to evade inactivation of ODN by 5′-exonuclease.

10. The ODN does not contain a CG or a GGGG sequence.

11. The ODN has no significant homology with any part of the Genebank aside from IL-15.

12. The ODN does not form a hairpin structure on account of weak intrinsic base complementation.

ODN Inhibitory Activity:

The ODN was assessed in its non-stabilized form (D-oligo) in a single experiment, in which human renal proximal tubular cells were incubated for 48 hours with 2 M ODN (double administration, 24 hours apart). The ODN inhibited inducible IL-15 production by 23.41%, and basal IL-15 production by 11.31%.

ODN 4.

Sequence of ODN: ′5-TCA TAC TCA AAG CCA CGG TA-′3 (SEQ. ID. NO. 6)

Length of ODN: 20 nucleotides.

Target location of ODN on IL-15-mRNA (base numbering by Genebank): 300-319

Rational Behind ODN Selection:

1. The ODN is targeted to the 5′-UTR of the IL-15-mRNA.

2. The ODN is targeted to the AUG start codon in the 5′-UTR of the IL-15-mRNA.

3. The computed structure of the IL-15-mRNA in this region is a single-strand loop positioned on a double-strand stem, separated from neighboring mRNA structures. This structure is identical in all IL-15-mRNA configurations but three.

4. The part of ODN that is aimed for IL-15-mRNA single-strand binding is longer than four nucleotides, and is positioned away from the 5′ end of the ODN, to evade inactivation of ODN by 5′-exonuclease.

5. The ODN does not contain a CG or a GGGG sequence.

6. The ODN has no significant homology with any part of the Genebank aside from IL-15.

7. The ODN does not form a hairpin structure on account of weak intrinsic base complementation. ODN inhibitory activity:

The ODN was assessed in its non-stabilized form (D-oligo) in three separate experiments. In each experiment, human renal proximal tubular cells were incubated for 48 hours with 2 μM ODU (double administration, 24 hours apart). The ODN inhibited inducible IL-15 production by 27.8%±6.1, and basal IL-15 production by 20.0%±2.9.

The specificity of action of the ODN was demonstrated by modifying the above experiment: IL-1 rather than IFNγ was used as the stimulant, and IL-6 levels were determined. The results indicate that IL-6 induction by IL-1 is unaffected by the ODN administration.

ODN 5.

Sequence of ODN: ′5-AAA TAC TTC TCA AAT GTG GT-′3 (SEQ. ID. NO. 7)

Length of ODN: 20 nucleotides.

Target location of ODN on IL-15-mRNA (base numbering by Genebank): 331-350

Rational Behind ODN Selection:

1. The ODN is targeted to the ORF of the IL-15-mRNA.

2. The computed structure of the IL-15-mRNA in this region is a single-strand loop positioned on a double-strand stem, separated from neighboring mRNA structures. This structure is identical in all IL-15-mRNA configurations but three.

3. The part of ODN that is aimed for IL-15-mRNA single-strand binding is longer than four nucleotides, and is positioned away from the 5′end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 43.94%, and basal IL-15 production by 33.58%.

The specificity of action of the ODN was demonstrated by modifying the above experiment: IL-1 rather IFNγ was used as the stimulant, and IL-6 levels were determined. The results indicate that IL-6 induction by IL-1 is unaffected by the ODN administration.

ODN 6.

Sequence of ODN: ′5-CAT CAC TTT CCG TAT ATA AA-′3 (SEQ. ID. NO. 8)

Length of ODN: 20 nucleotides.

Target location of ODN on IL-15-mRNA (base numbering by Genebank): 532-551

Rational Behind ODN Selection:

1. The ODN is targeted to the ORF of the IL-15-mRNA.

2. The computed structure of the IL-15-mRNA in this region is comprised of two single-strand loops in tandem, positioned on a double-strand stem and separated from neighboring mRNA structures. This structure is identical in all IL-15-mRNA configurations but three.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in two separate experiments. In each experiment, human renal proximal tubular cells were incubated for 48 hours with 2 M ODN (double administration, 24 hours apart). The ODN inhibited inducible IL-15 production by 11.72%±0.05, and basal IL-15 production by 22.83%±4.9.

ODN 7.

Sequence of ODN: ′5-AAA ACT CTG CAA AAA TTC TT-′3 (SEQ. ID. NO. 9)

Length of ODN: 20 nucleotides.

Target location of ODN on IL-15-mRNA (base numbering by Genebank): 750-769

Rational Behind ODN Selection:

1. The ODN is targeted to the ORF of the IL-15-mRNA.

2. The computed structure of the IL-15-mRNA in this region is composed of two single-strand loops in tandem, positioned on a double-strand stem and separated from neighboring mRNA structures. This structure is identical in all IL-15-mRNA configurations but five.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hair-pin structure on account of weak intrinsic base complementation.

ODN Inhibitory Activity:

The ODN was assessed in its non-stabilized form (D-oligo) in seven separate experiments. In each experiment, human renal proximal tubular cells were incubated for 48 hours with 2 M ODN (double administration, 24 hours apart). The ODN inhibited inducible IL-15 production by 30.83%±4.55, and basal IL-15 production by 27.28%±4.47.

ODN 8.

Sequence of ODN: ′5-AGT GAA ATA ACT TGT AAC TC-′3 (SEQ. ID. NO. 10)

Length of ODN: 20 nucleotides.

Target location of ODN on IL-15-mRNA (base numbering by Genebank): 596-615

Rational Behind ODN Selection:

1. The ODN is targeted to the ORF of the IL-15-mRNA.

2. The computed structure of the IL-15-mRNA in this region is composed of two single-strand loops in tandem, positioned on a double-strand stem and separated from neighboring mRNA structures. This structure is identical in all IL-15-mRNA configurations.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hair-pin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 7.0%, and basal IL-15 production by 21.42%.

ODN 9.

Sequence of ODN: ′5-TCA GAT TTT CTA CTG TAT CA-′3 (SEQ. ID. NO. 11)

Length of ODN: 20 nucleotides.

Target location of ODN on IL-15-mRNA (base numbering by Genebank): 640-659

Rational Behind ODN Selection:

1. The ODN is targeted to the ORF of the IL-15-mRNA.

2. The computed structure of the IL-15-mRNA in this region is composed of two single-strand loops in tandem, positioned on a double-strand stem and separated from neighboring mRNA structures. This structure is identical in all IL-15-mRNA configurations but four, in all of whom the structure is one long single-strand loop.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 25.41%, and basal IL-15 production by 26.52%.

ODN 10.

Sequence of ODN: ′5-GAG TTC ATC TGA TCC AAG GT-′3 (SEQ. ID. NO. 12)

Length of ODN: 20 nucleotides.

Target location of ODN on IL-15-mRNA (base numbering by Genebank): 933-952

Rational Behind ODN Selection:

1. The ODN is targeted to the 3′-UTR of the IL-15-mRNA.

2. The computed structure of the IL-15-mRNA in this region is composed of two single-strand loops in tandem, positioned on a double-strand stem and separated from neighboring mRNA structures. This structure is identical in all IL-15-mRNA configurations but two, one of which is a single loop structure, and the other a triple loop structure.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hair-pin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 9.97%, and basal IL-15 production by 9.44%.

ODN 11.

Sequence of ODN: ′5-AAG TAA GTC TGA GAG AAG TT-′3 (SEQ. ID. NO. 13)

Length of ODN: 20 nucleotides.

Target location of ODN on IL-15-mRNA (base numbering by Genebank): 1000-1019

Rational Behind ODN Selection:

1. The ODN is targeted to the 3′-UTR of the IL-15-mRNA, adjacent upstream to an AUUUA motif.

2. The computed structure of the IL-15-mRNA in this region is composed of two single-strand loops in tandem, positioned on a double-strand stem and separated from neighboring mRNA structures. This structure is identical in all IL-15-mRNA configurations but two, one of which is a single loop structure.

3. The part of ODN that is aimed for IL-15-mRNA single-strand binding is longer than four nucleotides, and is positioned away from the ′5 end of the ODN, to evade inactivation of ODN by ′5-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 21.3%, and basal IL-15 production by 13.5%.

ODN 12.

Sequence of ODN: ′5-TCA ACA TTT TAC ATT ATT CC-′3 (SEQ. ID. NO. 14)

Length of ODN: 20 nucleotides.

Target location of ODN on IL-15-mRNA (base numbering by Genebank): 1062-1081

Rational Behind ODN Selection:

1. The ODN is targeted to the 3′-UTR of the IL-15-mRNA, adjacent downstream to an AUUUA motif.

2. The computed structure of the IL-15-mRNA in this region is composed of two single-strand loops in tandem, positioned on a double-strand stem and separated from neighboring mRNA structures. This structure is identical in all IL-15-mRNA configurations.

3. The part of ODN that is aimed for IL-15-mRNA single-strand binding is longer than four nucleotides, and is positioned away from the 5′ end of the ODN, to evade inactivation of ODN by 5′-exonuclease.

4. The ODN does not contain a CG or a GGGG sequence.

5. The ODN has no significant homology with any part of the Genebank aside from IL-15.

6. The ODN does not form a hairpin structure on account of weak intrinsic base complementation.

ODN Inhibitory Activity:

The ODN was assessed in its non-stabilized form (D-oligo) in one experiment, in which human renal proximal tubular cells were incubated for 48 hours with 2 M ODN (double administration, 24 hours apart). The ODN inhibited inducible IL-15 production by 14.2%, and basal IL-15 production by 10.1%.

Combinatory Antisense ODN Preparations

Combination #1:

Sequence of ODN-1: ′5-ATA TGT ACA ATT TCA ATA AT-′3 (SEQ. ID. NO. 2)

Sequence of ODN-7: ′5-AAA ACT CTG CAA AAA TTC TT-′3 (SEQ. ID. NO. 9)

Rational Behind ODN Pairing:

1. ODN-1 is targeted to the ′3-UTR of IL-15-mRNA while ODN-7 is targeted to the ORF of IL-15-mRNA. This distinction between target sites most likely provides the two ODN molecules with differing mechanisms of IL-15 production inhibition. Such a polarity can promote a synergistic relationship between the two, achieving a higher inhibitory activity in the combination mixture than what each individual ODN would achieve alone, at the same total concentration.

2. The two do not hybridize with each other.

3. The two have been screened for incompatibility, as described above in ‘individual ODN preparations’.

Inhibitory Effect of ODN Combination:

The combination was tested in eight separate experiments. Each experiment is identical to the individual-ODN experiments, as described above, with the exception of using 1 M from each ODN, totaling 2 M ODN. The results indicated an inhibition of 59.6%±14.5 of inducible IL-15 production, and 50.4%±9.0 of basal IL-15 production.

Combination #2:

Sequence of ODN-5: ′5-AAA TAC TTC TCA AAT GTG GT-′3 (SEQ. ID. NO. 7)

Sequence of ODN-6: ′5-CAT CAC TTT CCG TAT ATA AA-′3 (SEQ. ID. NO. 8)

Rational Behind ODN Pairing:

1. ODN-5 and ODN-6 are both targeted to the ORF of IL-15-mRNA, but are positioned on dissimilar and distant structures. This distinction between target sites most probably promotes an additive relationship between the two, though there is a possibility that enhanced disfiguring of IL-15-mRNA by extensive hybridization with ODN molecules destabilizes the molecule, and achieves a higher inhibitory effect.

2. The two do not hybridize with each other.

3. The two have been screened for incompatibility, as described above in ‘individual ODN preparations’.

Inhibitory Effect of ODN Combination:

The combination was tested in one experiment. The experiment is identical to the individual-ODN experiments, as described above, with the exception of using 1 M from each ODN, totaling 2 M ODN. The results indicated an inhibition 27.22% of inducible IL-15 production, and 25.24% of basal IL-15 production.

Combination #3:

Sequence of ODN-7: ′5-AAA ACT CTG CAA AAA TTC TT-′3 (SEQ. ID. NO. 9)

Sequence of ODN-4: ′5-TCA TAC TCA AAG CCA CGG TA-′3 (SEQ. ID. NO. 6)

Rational Behind ODN Pairing:

1. ODN-7 and ODN-4 are both targeted to the ORF of IL-15-mRNA, but are positioned on dissimilar and distant structures. This distinction between target sites most probably promotes an additive relationship between the two, though there is a possibility that enhanced disfiguring of IL-15-mRNA by extensive hybridization with ODN molecules destabilizes the molecule, and achieves a higher inhibitory effect.

2. The two do not hybridize with each other.

3. The two have been screened for incompatibility, as described above in ‘individual ODN preparations’.

Inhibitory Effect of ODN Combination:

The combination was tested in three separate experiments. The experiments are identical to the individual-ODN experiments, as described above, with the exception of using 1 M from each ODN, totaling 2 M ODN. The results indicated an inhibition 46.7%±4.4 of inducible IL-15 production, and 60.5%±19.1 of basal IL-15 production.

Combination #4:

Sequence of ODN-11: ′5-AAG TAA GTC TGA GAG AAG TT-′3 (SEQ. ID. NO. 13)

Sequence of ODN-7: ′5-AAA ACT CTG CAA AAA TTC TT-′3 (SEQ. ID. NO. 9)

Rational Behind ODN Pairing:

1. ODN-11 is targeted to the ′3-UTR of IL-15-mRNA while ODN-7 is targeted to the ORF of IL-15-mRNA. This distinction between target sites most likely provides the two ODN molecules with differing mechanisms of IL-15 production inhibition. Such a polarity can promote a synergistic relationship between the two, achieving a higher inhibitory activity to the combination mixture than each ODN, at the same total concentration, would achieve alone.

2. The two do not hybridize with each other.

3. The two have been screened for incompatibility, as described above in ‘individual ODN preparations’.

Inhibitory Effect of ODN Combination:

The combination was tested in one experiment. The experiment is identical to the individual-ODN experiments, as described above, with the exception of using 1 M from each ODN, totaling 2 M ODN. The results indicated an inhibition 31.66% of inducible IL-15 production, and 21.88% of basal IL-15 production.

Combination #5:

Sequence of ODN-12: ′5-TCA ACA TTT TAC ATT ATT CC-′3 (SEQ. ID. NO 14) Sequence of ODN-7: ′5-AAA ACT CTG CAA AAA TTC TT-′3 (SEQ. ID. NO. 9)

Rational Behind ODN Pairing:

1. ODN-12 is targeted to the ′3-UTR of IL-15-mRNA while ODN-7 is targeted to the ORF of IL-15-mRNA. This distinction between target sites most likely provides the two ODN molecules with differing mechanisms of IL-15 production inhibition. Such a polarity can promote a synergistic relationship between the two, achieving a higher inhibitory activity to the combination mixture than each ODN, at the same total concentration, would achieve alone.

2. The two do not hybridize with each other.

3. The two have been screened for incompatibility, as described above in ‘individual ODN preparations’.

Inhibitory Effect of ODN Combination:

The combination was tested in one experiment. The experiment is identical to the individual-ODN experiments, as described above, with the exception of using 1 mM from each ODN, totaling 2 mM ODN. The results indicated an inhibition 40.0% of inducible IL-15 production, and 27.58% of basal IL-15 production.

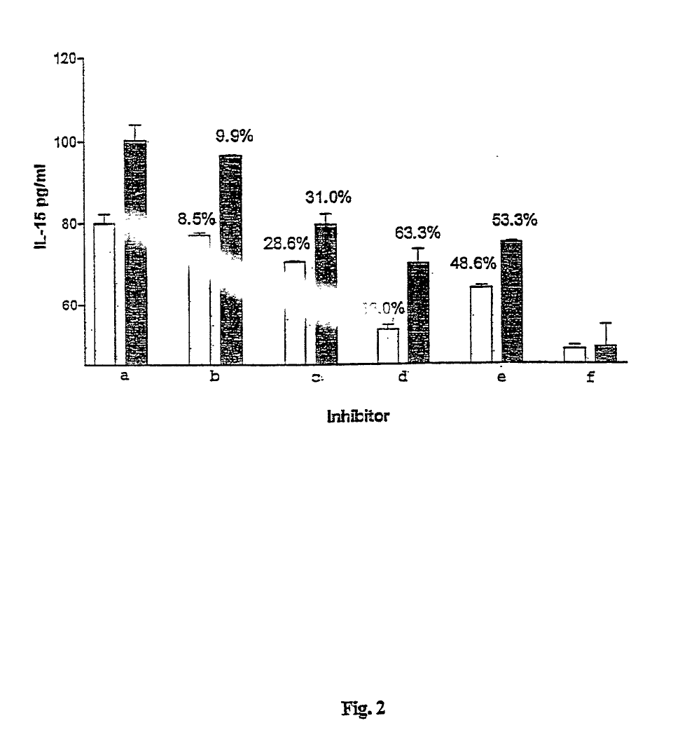

FIG. 2 shows the results of some of the above experiments: a is an experiment without any addition of ODN, b is with ODN 1, c with ODN 7, d and e with both ODN 1 and ODN 7, and f with cycloheximide. b, c, and d used a total of 2 μM ODN, while e used a total of 1 μM ODN. The left, empty bar represents basal IL-15 production, while the right, filled-in bar represents IFN-γ induced production. The percentages over the bars indicate inhibition of IL-15 production, with a being equivalent to 0% inhibition, and f representing 100% inhibition.

1. An antisense oligomer capable of inhibiting production of interleukin-15 (IL-15) by hybridizing to the 5′-UTR or 3′-UTR regions of the mRNA of IL-15.

2. An oligomer according to claim 1 selected from the group consisting of ODN 1 through ODN 12.

3. An oligomer according to claim 1 which is selected from the group consisting of an oligonucleotide and an oligonucleotide analog.

4. An oligomer according to claim 3, wherein the oligonucleotide is DNA.

5. An oligonucleotide according to claim 3 wherein the oligonucleotide is selected from the group consisting of phosphodiester, phosphorothioate, ethylphosphonate, methylphosphonate, and methylphosphonothioate oligonucleotides.

6. An oligomer according to claim 3, wherein the oligonucleotide is at least 5 nucleotides in length.

7. An oligomer according to claim 6, wherein the length is from 5 to 50 nucleotides.

8. An oligomer according to claim 3, wherein the oligonucleotide analog is selected from the group consisting of protein nucleic acid morpholino, methylene linkage, boronated, and pteridine oligonucleotide analogs.

9. An oligomer according to claim 3, wherein the oligonucleotide analog is linked at its 5′ end or at its 3′ end to an intercalator.

10. An oligomer according to claim 9, wherein the intercalator is selected from the group consisting of psoralen and acridine derivatives.

11. An oligomer comprising an oligomer according to claim 1.

12. An oligomer according to claim 1, wherein the oligomer is capable of alleviating inflammatory polyarthopathy.

13. An oligomer according to claim 12, wherein the inflammatory polyarthopathy is associated with rheumatoid arthritis, organ and cell transplant rejections and inflammatory bowel disease.

14. An oligomer according to claim 13 wherein said organ transplant rejection is a kidney transplant rejection.

15. A method of inhibiting production of interleukin-15 (IL-15) by a cell comprising the steps of:
(One) introducing an antisense oligomer according to claim 1 into said cell; and
(Two) allowing said oligomer to hybridize to the mRNA of IL-15, thereby inhibiting the production of IL-15.

16. A method according to claim 15, wherein the oligomer is dispersed in a pharmaceutically acceptable carrier.

17. A method according to claim 16, wherein the carrier is selected from the group consisting of injectable preparations, sprays, ointments, creams, gels, tablets, and perfusions.

18. A method according to claim 15, wherein the oligomer is selected from the group consisting of an oligonucleotide and an oligonucleotide analog.

19. A method according to claim 18, wherein the oligonucleotide analog is selected from the group consisting of protein nucleic acid, morpholino, methylene linkage, boronated, and pteridine oligonucleotide analogs.

20. A method according to claim 18, wherein the oligonucleotide analog is linked at its 5′ end or its 3′ end to an intercalator.

21. A method according to claim 20, wherein the intercalator is selected from the group consisting according to psoralen and acridine derivatives.

22. A method according to claim 18, wherein the oligonucleotide is DNA.

23. A method according to claim 22, wherein the oligonucleotide is selected from the group consisting of phosphodiester, phosphorothioate, methylphosphonate, and methylphosphonothioate oligonucleotides.

24. A method according to claim 18, wherein the oligonucleotide is at least 5 nucleotides in length.

25. A method according to claim 24, wherein the length is from 5 to 50 nucleotides.

26. A method according to claim 15 wherein a combination of two or more different said oligomers are introduced into said cell.

27. A pharmaceutical composition for treating diseases associated with the production of IL-15 comprising an antisense oligomer capable of inhibiting production of interleukin-15 (IL-15) by hybridizing to the 5′-UTR or 3′-UTR regions of the mRNA of IL-15, and a pharmaceutically acceptable carrier.

28. A pharmaceutical composition according to claim 27 wherein said antisense oligomer is selected from the group consisting of ODN 1 (SEQ. ID. NO 2), ODN2 (SEQ. ID. NO 4), ODN3 (SEQ . ID. NO 5), ODN 4 (SEQ. ID. NO 6), ODN 5 (SEQ. ID. NO. 7), ODN 6 (SEQ. ID. NO. 8), ODN 7 (SEQ. ID. NO. 9), ODN 8 (SEQ. ID. NO. 10), ODN 9 (SEQ. ID. NO. 11), ODN 10 (SEQ. ID. NO. 12), ODN 11 (SEQ. ID. NO. 13) and ODN 12 (SEQ. ID. NO. 14).

29. A pharmaceutical composition according to claim 27 wherein said oligomer is selected from the group consisting of an oligonucleotide and an oligonucleotide analog.

30. A pharmaceutical composition according to claim 29 wherein the oligonucleotide is DNA.

31. A pharmaceutical composition according to claim 29, wherein the oligonucleotide is selected from the group consisting of phosphodiester, phosphorothioate, ethylphosphonate, methylphosphonate, and methylphosphonothioate oligonucleotides.

32. A pharmaceutical composition according to claim 29 wherein the oligonucleotide is at least 5 nucleotides in length.

33. A pharmaceutical composition according to claim 32, wherein the length is from 5 to 50 nucleotides.

34. A pharmaceutical composition according to claim 28, wherein the oligonucleotide analog is selected from the group consisting of protein nucleic acid, morpholino, methylene linkage, boronated, and pteridine oligonucleotide analogs.

35. A pharmaceutical composition according to claim 28, wherein the oligonucleotide analog is linked at its 5′ end or its 3′ end to an intercalator.

36. A pharmaceutical composition according to claim 34, wherein the intercalator is selected from the group consisting of psoralen and acridine derivatives.

37. A pharmaceutical composition according to claim 27 comprising a combination of two or more different said oligomers.

38. A pharmaceutical composition according to claim 27, wherein the carrier is selected from the group consisting of injectable preparations, sprays, ointments, creams, gels, tablets, and perfusions.

39. A pharmaceutical composition according to claim 27 wherein said disease is an inflammatory polyarthopathy associated with rheumatoid arthritis, organ and cell transplant rejections, asthma, lupus erythematosus and inflammatory bowel disease.

40. A pharmaceutical composition according to claim 39 wherein said organ transplant rejection is a kidney transplant rejection.


SEQUENCE LISTING (14 sequences)

SEQ ID NO: 1 - DNA, 1202 bp, Homo sapiens
tgtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttaaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgct

SEQ ID NO: 2 - DNA, 20 bp, Homo sapiens
atatgtacaatttcaataat

SEQ ID NO: 3 - DNA, 20 bp, Homo sapiens
atatgtaggcattcaataat

SEQ ID NO: 4 - DNA, 20 bp, Homo sapiens
aagagccacatggccatata

SEQ ID NO: 5 - DNA, 20 bp, Homo sapiens
cattgatttctgtgctccat

SEQ ID NO: 6 - DNA, 20 bp, Homo sapiens
tcatactcaaagccacggta

SEQ ID NO: 7 - DNA, 20 bp, Homo sapiens
aaatacttctcaaatgtggt

SEQ ID NO: 8 - DNA, 20 bp, Homo sapiens
catcactttccgtatataaa

SEQ ID NO: 9 - DNA, 20 bp, Homo sapiens
aaaactctgcaaaaattctt

SEQ ID NO: 10 - DNA, 20 bp, Homo sapiens
agtgaaataacttgtaactc

SEQ ID NO: 11 - DNA, 20 bp, Homo sapiens
tcagattttctactgtatca

SEQ ID NO: 12 - DNA, 20 bp, Homo sapiens
gagttcatctgatccaaggt

SEQ ID NO: 13 - DNA, 20 bp, Homo sapiens
aagtaagtctgagagaagtt

SEQ ID NO: 14 - DNA, 20 bp, Homo sapiens
tcaacattttacattattcc